(19) United States
(12) Patent Application Publication
(10) Pub. No.: US 2025/0381409 A1
(43) Pub. Date: Dec. 18, 2025

(54) RECEIVER CONTROLLED VARIABLE INDUCTIVE TRANSMIT OUTPUT

(71) Applicant: Cardiac Pacemakers, Inc., St. Paul, MN (US)

(72) Inventors: Daniel Joseph Landherr, Wyoming, MN (US); Jason Lahr, Coon Rapids, MN (US)

(21) Appl. No.: 19/216,424

(22) Filed: May 22, 2025

Related U.S. Application Data
(60) Provisional application No. 63/659,694, filed on Jun. 13, 2024.

(51) Int. Cl. A61N 1/372; G16H 40/67

(52) U.S. Cl. CPC A61N 1/37223; G16H 40/67

(57) ABSTRACT

Systems and methods are disclosed for inductive telemetry of an implantable medical device including a receiver circuit configured to receive an input signal from a telemetry coil of the implantable medical device and a transmitter circuit configured to selectively drive the telemetry coil of the implantable medical device in a selective one of a single-ended mode or a differential mode using information from the receiver circuit.

### CLAIM OF PRIORITY

This application claims the benefit of U.S. Provisional Application No. 63/659,694, filed on Jun. 13, 2024, which is hereby incorporated by reference in its entirety.

### TECHNICAL FIELD

This document relates generally to medical devices and more particularly to variable inductive telemetry transmitter output for an implantable medical device.

### BACKGROUND

Ambulatory medical devices, including implantable, subcutaneous, wearable, insertable, or one or more other medical devices, etc., can monitor, detect, or treat various conditions, including heart failure (HF), atrial fibrillation (AF), etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc. Frequent patient monitoring can provide early detection of worsening patient condition, including worsening heart failure or atrial fibrillation.

Ambulatory patient monitoring can provide early detection of worsening patient condition, including worsening heart failure or atrial fibrillation. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient status, including improving or worsening patient condition, and modifying one or more medical device functions based thereon, can improve medical device efficiency, such as by reducing unnecessary resource consumption, thereby extending the usable life of the ambulatory medical device.

### SUMMARY

Systems and methods are disclosed for inductive telemetry of an implantable medical device including a receiver circuit configured to receive an input signal from a telemetry coil of the implantable medical device and a transmitter circuit configured to selectively drive the telemetry coil of the implantable medical device in a selective one of a single-ended mode or a differential mode using information from the receiver circuit.

An example of subject matter (e.g., an implantable medical device system configured for inductive telemetry) may comprise means for receiving an input signal from a telemetry coil and means for selectively driving the telemetry coil in one of a single-ended mode or a differential mode using information about the input signal.

In an example, which may be combined with any one or more examples described herein, the means for receiving the input signal comprises a receiver circuit configured to receive the input signal from the telemetry coil and the means for selectively driving the telemetry coil in one of the single-ended mode or the differential mode using information about the input signal comprises a transmitter circuit having the single-ended mode and the differential mode, wherein the transmitter circuit is configured to selectively drive the telemetry coil in one of the single-ended mode or the differential mode using information from the receiver circuit.

An example of subject matter (e.g., an implantable medical device system configured for inductive telemetry) may comprise a receiver circuit configured to receive an input signal from a telemetry coil and a transmitter circuit having a single-ended mode and a differential mode, wherein the transmitter circuit is configured to selectively drive the telemetry coil in one of the single-ended mode or the differential mode using information from the receiver circuit.

In an example, which may be combined with any one or more examples described herein, the subject matter may comprise a control circuit configured to determine a mode of operation for the transmitter circuit using information from the receiver circuit, and the transmitter circuit may optionally be configured to drive the telemetry coil in one of the single-ended mode or the differential mode based on the determined mode of operation.

In an example, which may be combined with any one or more examples described herein, the transmitter circuit is configured to drive the telemetry coil in the single-ended mode if the information from the receiver circuit indicates that a distance of the telemetry coil from an external telemetry coil does not exceed a threshold distance and to drive the telemetry coil in the differential mode if the information from the receiver circuit indicates that a distance of the telemetry coil from an external telemetry coil exceeds a threshold distance.

In an example, which may be combined with any one or more examples described herein, the receiver circuit includes a comparator having a dynamic range and one of an amplifier or a resistor network and the receiver circuit is configured to determine an attenuation or gain setting for the receiver circuit to apply to the input signal from the telemetry coil using the amplifier or the resistor network to keep an attenuated or amplified input signal in the dynamic range of the comparator.

In an example, which may be combined with any one or more examples described herein, the dynamic range of the comparator has high and low thresholds, the receiver circuit is configured to adjust the attenuation or gain setting for the receiver circuit using the attenuated or amplified input signal exceeding the high threshold or falling below the low threshold, the attenuation or grain setting comprises at least two settings, a first setting indicating more attenuation and a second indicating less attenuation, and the transmitter circuit is configured to drive the telemetry coil in the single-ended mode in the first setting and in the differential mode in the second setting.

In an example, which may be combined with any one or more examples described herein, the transmitter circuit is configured to transition from the single-ended mode to the differential mode, or from the differential mode to the single-ended mode, based on information from the receiver circuit.

In an example, which may be combined with any one or more examples described herein, to drive the telemetry coil in the single-ended mode comprises to drive the telemetry coil between a supply voltage of the implantable medical device and ground and to drive the telemetry coil in the differential mode comprises to drive the telemetry coil between a supply voltage of the implantable medical device and a negative supply voltage of the implantable medical device.

In an example, which may be combined with any one or more examples described herein, the information from the receiver circuit includes a current or a voltage produced by the telemetry coil in response to an applied electromagnetic field by an external telemetry coil.

In an example, which may be combined with any one or more examples described herein, the subject matter may optionally comprise a telemetry block including the receiver circuit, the transmitter circuit, and the telemetry coil, wherein the telemetry coil comprises a coil of wire having a number of turns and the telemetry block is configured to communicate digital data between the implantable medical device and a remote patient management system comprising an external telemetry coil.

An example of subject matter (e.g., method for inductive telemetry in an implantable medical device) may comprise receiving an input signal from a telemetry coil using a receiver circuit and selectively driving the telemetry coil, using a transmitter circuit having a single-ended mode and a differential mode, in a selective one of the single-ended mode or the differential mode using information from the receiver circuit.

In an example, which may be combined with any one or more examples described herein, the subject matter may optionally comprise determining a mode of operation for the transmitter circuit using information from the receiver circuit, wherein selectively driving the telemetry coil includes driving the telemetry coil in one of the single-ended mode or the differential mode based on the determined mode of operation.

In an example, which may be combined with any one or more examples described herein, selectively driving the telemetry coil includes driving the telemetry coil in the single-ended mode if the information from the receiver circuit indicates that a distance of the telemetry coil from an external telemetry coil does not exceed a threshold distance and driving the telemetry coil in the differential mode if the information from the receiver circuit indicates that a distance of the telemetry coil from an external telemetry coil exceeds a threshold distance.

In an example, which may be combined with any one or more examples described herein, the subject matter may optionally comprise determining an attenuation or gain setting for the receiver circuit to apply to the input signal from the telemetry coil using one of an amplifier or a resistor network to keep an attenuated or amplified input signal in a dynamic range of a comparator of the receiver circuit.

In an example, which may be combined with any one or more examples described herein, the subject matter may optionally comprise adjusting the attenuation or gain setting for the receiver circuit using the attenuated or amplified input signal exceeding a high threshold or falling below a low threshold, wherein selectively driving the telemetry coil includes driving the telemetry coil in the single-ended mode in a first attenuation or grain setting indicating more attenuation and in the differential mode in a second attenuation or grain setting indicating less attenuation.

In an example, which may be combined with any one or more examples described herein, the subject matter may optionally comprise transitioning the transmitter circuit from the single-ended mode to the differential mode, or from the differential mode to the single-ended mode, using information from the receiver circuit.

In an example, which may be combined with any one or more examples described herein, driving the telemetry coil in the single-ended mode comprises driving the telemetry coil between a supply voltage of the implantable medical device and ground and driving the telemetry coil in the differential mode comprises driving the telemetry coil between the supply voltage of the implantable medical device and a negative supply voltage of the implantable medical device.

In an example, which may be combined with any one or more examples described herein, the subject matter may optionally comprise communicating digital data in an inductive telemetry session between the implantable medical device and a remote patient management system, wherein the implantable medical device comprises a telemetry block including the receiver circuit, the transmitter circuit, and the telemetry coil, wherein the telemetry coil comprises a coil of wire having a number of turns, wherein the remote patient management system comprises an external telemetry coil, and wherein the information from the receiver circuit includes a current or a voltage produced by the telemetry coil in response to an applied electromagnetic field by the external telemetry coil.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example system including an implantable medical device and a remote patient management device in inductive communication.

FIG. 2 illustrates an example of a receiver circuit.

FIGS. 3 and 4 illustrate example methods.

FIG. 5 illustrates an example medical device system.

FIG. 6 illustrates an example patient management system and portions of an environment in which the system may operate.

FIGS. 7-9 illustrate example implantable medical devices.

FIG. 10 illustrates an example remote patient management system.

FIG. 11 illustrates an example machine upon which any one or more of the techniques discussed herein may perform.

### DETAILED DESCRIPTION

Ambulatory medical devices can be implanted in or otherwise positioned on or about patients to monitor physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) cardiac rhythm management devices configured to monitor or provide stimulation to the patient.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICDs), subcutaneous implantable cardioverter defibrillators (S-ICDs), cardiac resynchronization therapy defibrillators (CRT-Ds), insertable cardiac monitors (ICMs), leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (LV) (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Ambulatory medical devices, including implantable cardiac rhythm management, cardiac resynchronization therapy, or monitoring devices, etc., include wireless telemetry circuits and components to communicate via one or both of radio frequency (RF) telemetry or inductive telemetry with one or more other ambulatory medical or external devices, such as a remote patient management device, to enable data transfer from or remote programming of the ambulatory medical device in one or more telemetry sessions. RF telemetry (e.g., short-range RF telemetry), such as Medical Implant Communication Service (MICS) (402-405 MHz frequency with a range of 2 m); Bluetooth® or Bluetooth® Low Energy (BLE) (2.4-2.483 GHZ frequency with a range up to 10 m), etc., utilizes radio waves to communicate over distances up to several meters or more. In contrast, inductive telemetry utilizes electromagnetic induction to communicate over short distances, typically less than 15 cm, minimizing interferences but requiring proximity between coupled telemetry antennas.

Existing external telemetry systems, such as existing external telemetry wands and telemetry circuits of remote patient management devices, are configured for communication at traditional distances (e.g., up to 6 cm) between respective telemetry coils of an external telemetry wand and a corresponding telemetry coils of implantable medical devices implanted at traditional subcutaneous positions in an upper thorax of the patient, above a pectoral muscle and below a clavicle of the patient. For different implant sites having deeper implant requirements, for example, greater than 9 cm or 10 cm, such as for leadless pacemakers positioned in a heart or coronary vasculature, subcutaneous implantable cardioverter defibrillators implanted at a lateral thoracic region, etc., the transmit power to the telemetry coil of the implantable medical device can be increased. However, an increase in transmit power may saturate or overwhelm existing external telemetry systems (e.g., having a fixed or limited gain, etc.) at close distances (e.g., at or near 0 cm, etc.).

Accordingly, the present inventors have recognized, among other things, systems and methods to control a variable inductive transmitter output of an inductive telemetry block of an implantable medical device, for example, to a telemetry coil of the implantable medical device, such as by using information from a receiver circuit of the inductive telemetry block, to accommodate different implant locations and depths of the implantable medical device with respect to a patient, in certain examples, without requiring changes in an external telemetry wand or telemetry circuits of an existing remote patient management device configured for inductive communication with the implantable medical device over more traditional, shallower implant sites or shorter distances. For example, the telemetry coil of the implantable medical device can be selectively or variably driven single-ended or differentially to meet deeper implant performance requirements and also not overwhelm existing external telemetry wand or telemetry circuits at close distances.

Single-ended drive (e.g., driving the telemetry coil between a supply voltage and ground, etc.) conserves current draw and is sufficient for traditional, shorter distances (e.g., less than 6 cm, including at or near 0 cm) or shallower implant locations, such as for traditional tachycardia or bradycardia cardiac rhythm management devices, etc. Differential drive (e.g., driving the telemetry coil between a supply voltage and a negative supply voltage, etc.) can increase (e.g., effectively double) the transmit output power and current draw of the inductive telemetry block of the implantable medical device based on a supply voltage (e.g., a fixed supply voltage), extending the inductive telemetry range for deeper implant locations, such as subcutaneous implantable cardioverter defibrillators, etc. In an example, switching from single-ended to differential drive can be controlled based on a gain or attenuation setting of a receiver circuit of the inductive telemetry block of the implantable medical device, thereby enabling communication with an existing external telemetry wand at a larger range of distances without changing the design of the external telemetry wand or external telemetry circuits.

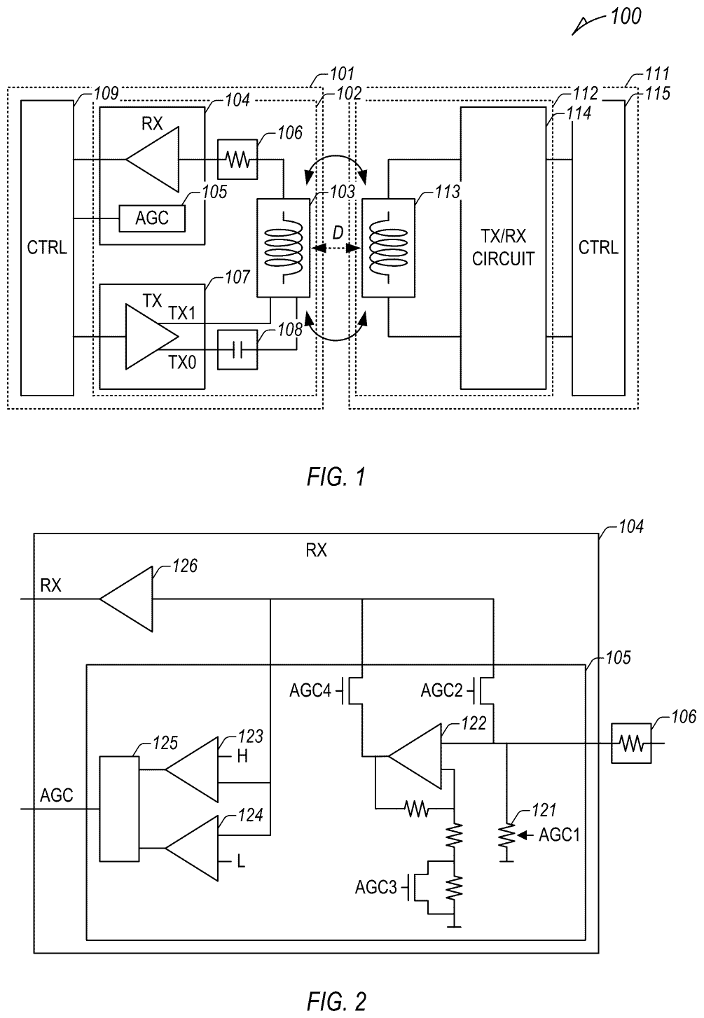

FIG. 1 illustrates an example system 100 including an implantable medical device 101 (e.g., a subcutaneous implantable cardioverter defibrillator, leadless cardiac pacemaker, etc.) and a remote patient management device 111 (e.g., LATITUDE™ Programming System, Model 3300, etc.) in inductive communication through respective telemetry coils (e.g., air-core antennas).

The implantable medical device 101 can include a telemetry block 102 including a telemetry coil 103 (e.g., a coil of wire having a number of turns, such as 90 turns of 41-gauge wire, etc.), a receiver circuit 104 (RX) (e.g., an analog receiver circuit), an automatic gain control (AGC) circuit 105, an attenuation resistor 106, a transmitter circuit 107 (TX), a capacitor 108, and a control circuit 109 coupled to the telemetry block 102. The telemetry coil 103, in combination with the capacitor 108, can form a tuned LC circuit that acts as one half of an air-coupled transformer with a corresponding telemetry coil of the remote patient management device 111. In an example, data can be transmitted from the implantable medical device 101 at a first frequency (e.g., a carrier frequency of 57 kHz, etc.) and received at a second frequency (e.g., 50 kHz, etc.). The transmitter circuit 107 can provide power amplification to drive the telemetry coil and the receiver circuit 104 can detect and convert input signals received at the telemetry coil 103 into digital data.

The remote patient management device 111 can include an external telemetry block 112, including an external telemetry coil 113 (e.g., a Model 6395 Telemetry Wand, etc.) and a transceiver circuit 114, and an external control circuit 115. In an example, the transceiver circuit 114 can have a fixed gain stage to ensure performance when a distance (D) between the telemetry coil 103 and the external telemetry coil 113 is large (e.g., at 6 cm or greater, etc.). However, with the fixed gain stage, if the coil voltage received at the external telemetry coil 113 is above an external telemetry block threshold, the transceiver circuit 114 can become overloaded. In certain examples, the external telemetry coil 113 includes separate transmit and receive coils, and the transceiver circuit 114 can include separate transmitter and receiver circuits, for example, for respective transmit and receive coils, etc.

In an example, the external telemetry block 112 can be configured to emit a series of pings at a specific repetition rate to wake the telemetry block 102 of the implantable medical device 101 for an inductive telemetry session. The receiver circuit 104 of the implantable medical device 101 can listen at specific intervals for an input signal from the telemetry coil 103, such as in response to an applied electromagnetic field, and use a current or a voltage of the input signal to determine one or more automatic gain control settings of the telemetry block 102 or the automatic gain control circuit 105 of the implantable medical device 101.

The automatic gain control circuit 105 can determine and provide a number of different attenuation or gain settings (e.g., between 4 and 1/32, doubling between steps, etc.) to the input signal from the telemetry coil 103, in certain examples, using an amplifier (with a selectable gain of 2, 4, etc.) or a voltage divider network (e.g., a resistor string having selectable values in combination with the attenuation resistor 106, etc.) to keep the attenuated or amplified input signal in the dynamic range of a comparator of the receiver circuit 104, such as defined by high and low thresholds of the comparator, etc.

The implantable medical device 101, having traditional implantable limitations with respect to power use and battery life, can have limited supply voltages (e.g., 1.25 V, 1.8 V, 2.0 V, etc.). The transmitter circuit 107 can drive the telemetry coil 103 (e.g., based on one of the limited supply voltages, such as the highest supply voltage) in one of two transmit modes: single-ended through a first transmitter output (TX0) in a first transmit mode, driving the telemetry coil 103 between the supply voltage (e.g., 2.0 V) and ground, to meet a first subset of distance requirements (e.g., 0 cm to 6 cm, within a distance threshold of 6 cm, etc.) and differentially through first and second transmitter outputs (driven oppositely through TX0 and TX1 at respective sides of the telemetry coil 103) in a second transmit mode, driving the telemetry coil 103 between the supply voltage (e.g., 2.0 V) and a negative supply voltage (e.g., −2.0 V, such as via switching), increasing the output signal of the telemetry coil 103 to effectively double the supply voltage, to meet a second subset of distance requirements (e.g., greater than 6 cm, exceeds the distance threshold of 6 cm, etc.) greater than the first subset.

TABLE 1
Example Transmit Mode Power

| Transmit | Avg | | Peak | Coil RMS | Pk-Pk Coil | RMS Coil |
|---|---|---|---|---|---|---|
| Mode | Current | | Current | Voltage | Voltage | Current |
| Differential | 19 | mA | 72 mA | 2.22 V | 9.9 V | 30 mA |
| Single-ended | 4.8 | mA | 37 mA | 1.12 V | 4.9 V | 15 mA |

Whereas a distance threshold is described in the previous example as above or below 6 cm, in certain examples, the distance threshold can effectively be more or less, with the first including 0 cm and the second at some distance greater than 0 cm (e.g., greater than 9 cm, greater than 10 cm, etc.). In an example, the transmit mode can be controlled by the attenuation or gain setting of the receiver circuit 104 or the automatic gain control circuit 105.

For example, an input signal having a smaller voltage is generally indicative of a larger distance between telemetry coils, whereas an input signal having a larger voltage is generally indicative of a smaller distance between telemetry coils. Accordingly, automatic gain control settings indicative of greater attenuation are generally indicative of a smaller distance between telemetry coils, whereas automatic gain control settings indicative of a smaller attenuation or gain are generally indicative of a larger distance between telemetry coils.

Example attenuation gain control settings are provided below in Table 2, including a plurality of automatic gain control states to attenuate or amplify the input signal received from the telemetry coil 103.

TABLE 2
Example Automatic Gain Control Settings

| AGC State | Atten | Gain | Net Gain |
|---|---|---|---|
| 0 | — | 4 | 4 |
| 1 | — | 2 | 2 |
| 2 | 1 | — | 1 |
| 3 | 1/2 | — | 0.5 |
| 4 | 1/4 | — | 0.25 |
| 5 | 1/8 | — | 0.125 |
| 6 | 1/16 | — | 0.0625 |
| 7 | 1/32 | — | 0.03125 |

In an example, the transmitter circuit 107 can provide single-ended drive in the first transmit mode if the automatic gain control settings are at or above a first threshold (e.g., indicating a distance between telemetry coils less than a threshold distance or a magnitude of an input signal above a threshold voltage) and differential drive in the second transmit mode if the automatic gain control settings are below the first threshold (e.g., indicating a distance between telemetry coils greater than a threshold distance or a magnitude of an input signal below a threshold voltage). In an example, the first threshold can include an automatic gain control state, such as 6 or one or more other states (e.g., 4, 5, 7, etc.).

In other examples, a distance or an indication of the distance between telemetry coils can be determined, such as by the receiver circuit 104, the transmitter circuit 107, the control circuit 109, or one or more other assessment circuits, etc., compared to a threshold, and used to control the transmit mode of the transmitter circuit 107. In other examples, the amplitude, magnitude, or voltage level of the input signal from the telemetry coil 103 can be determined, compared to a threshold, and used to control the transmit mode of the transmitter circuit 107.

In other examples, the control circuit 109 can be configured to attenuate an output of the transmitter circuit 107, such as using a resistor network (e.g., a resistor string, etc.) to reduce coil current, without altering the transmitter circuit 107. In other examples, one or more other supply voltages (e.g., 1.8 V, 2.2 V, etc.) can be used to drive the telemetry coil 103 based on information from the receiver circuit 104, such as the attenuation or gain setting of the receiver circuit 104 or the automatic gain control circuit 105, etc., as coil current of the telemetry coil 103 is related to supply voltage. Other combinations of altering the transmitter circuit 107 output signal, supply voltage, or attenuation are also possible based on information from the receiver circuit 104, such as the attenuation or gain setting of the receiver circuit 104 or the automatic gain control circuit 105, etc.

In an example, the automatic gain control circuit 105 can determine an attenuation or gain setting to apply to the input signal to keep the resulting signal between high and low thresholds (e.g., 130 mv and 50 mv respectively, etc.). If the value of the attenuated or amplified input signal is above the high threshold, gain can be reduced or attenuation can be increased or the attenuation or gain setting can correspondingly be decremented. If the value of the attenuated or amplified input signal is below the low threshold, gain can be increased or attenuation can be decreased or the attenuation or gain setting can correspondingly be incremented.

In certain examples, such as at idle or when no input signal is received by the telemetry coil 103, etc., the automatic gain control circuit 105 increments to maximum gain. The implantable medical device 101 can adjust the attenuation or gain settings for an inductive telemetry session quickly after several pings from the remote patient management device 111, but there could be several transmissions from the implantable medical device 101 that would use more power than necessary or not be heard/understood (would overwhelm) the external telemetry block 112. In an example, the telemetry block 102 can default to single-ended mode for a brief time (e.g., a settling time, etc.) to allow the automatic gain control circuit 105 to determine the appropriate attenuation or grain setting. Changes at the implantable medical device 101 can be limited to devices having a deeper implant requirement and can an existing remote patient management device 111 as well traditional implant requirement devices to remain in-use in the field without changes.

FIG. 2 illustrates an example of a receiver circuit 104, including a comparator 126, and an automatic gain control circuit 105. The automatic gain control circuit 105 can receive an input signal from a telemetry coil through an attenuation resistor 106 and provide the input signal to one or both of an attenuation circuit or an amplifier 122 depending on a desired attenuation or gain setting.

The attenuation circuit includes a selectable string of series resistors 121 that, in combination with the attenuation resistor 106, act as a selectable voltage divider depending on a first automatic gain control signal (AGC1). If no attenuation is desired, the series resistors 121 can remain inactive (open). When the attenuation circuit is selected, or when no attenuation is desired, a first switch is activated (closed) by a second automatic gain control signal (AGC2). The amplifier 122 includes a series of resistors to provide one or more selectable gain settings depending on, for example, a second switch activated (closed) by a third automatic gain control signal (AGC3). When amplification is desired, a third switch is activated (closed) by a fourth automatic gain control signal (AGC4). In certain examples, the series resistors 121 can terminate to ground, whereas the series of resistors coupled to the amplifier 122 can terminate to a common mode voltage (e.g., 50 mV, etc.) higher than ground, such as to account for voltage swing of the input signal from the telemetry coil.

The automatic gain control circuit 105 can additionally include comparators 123, 124 to compare a value of the input signal or an attenuated or amplified input signal to one or more thresholds, such as high and low thresholds. A logic circuit 125, such as a counter or other digital logic, can be used to determine an automatic gain control signal based on the comparisons. One or more other automatic gain control signals or states can be determined based on the comparisons, including a mode of operation of a transmitter circuit.

The comparator 126 can be configured to compare the input signal or the attenuated input signal to one or more thresholds, such as to produce a digital signal from the analog input signal from the telemetry coil, and in certain examples can include one or more of an analog to digital converter (ADC), a digital to analog converter (DAC) (e.g., such as to set one or more thresholds, etc.), or one or more other circuits to produce a digital output signal.

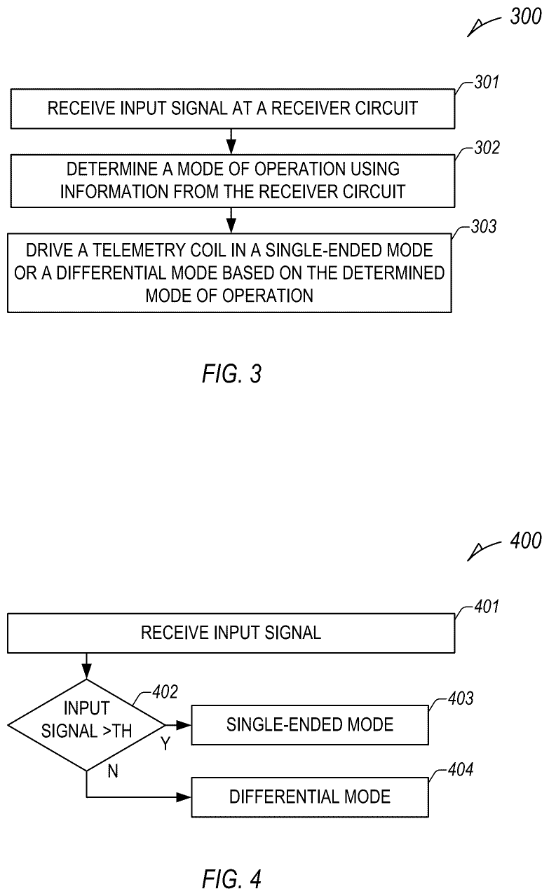

FIG. 3 illustrates an example method 300 for inductive telemetry in an implantable medical device. At step 301, an input signal is received, such as from a telemetry coil, using a receiver circuit of an implantable medical device. At step 302, a mode of operation is determined, for example, by a control circuit (e.g., a control circuit of the implantable medical device, an automatic gain control circuit, or one or more other control or assessment circuits, etc.), using information from the receiver circuit, such as an input signal, a value of the input signal (e.g., a voltage of the input signal, a current of the input signal, etc.), or a value of an attenuated or amplified input signal, etc. At step 303, the telemetry coil of the implantable medical device can be driven, such as by a transmitter circuit, in one of a single-ended mode or a differential mode based on the determined mode of operation.

In other examples, one or more automatic gain control settings can be determined using the input signal or the attenuated or amplified input signal, and the mode of operation can be determined based on one or more of the automatic gain control settings. For example, the automatic gain control settings can include a first attenuation setting indicating more attenuation (e.g., a greater reduction of the input signal from the telemetry coil) and a second attenuation setting indicating less attenuation (e.g., or no attenuation, some amplification, or more amplification, etc.). In an example, the telemetry coil of the implantable medical device can be driven in the single-ended mode in the first attenuation setting and in the differential mode in the second attenuation setting. Where there are more than two settings, a threshold can be selected to transition between the single-ended and differential modes. In addition, the transmitter circuit can transition from the single-ended mode to a differential mode or vice versa based on information from the receiver circuit, such as the determined mode, one or more automatic gain control settings, etc.

FIG. 4 illustrated an example method 400 for inductive telemetry in an implantable medical device. At step 401, an input signal is received, such as by a receiver circuit, a telemetry coil, or combinations thereof. At step 402, a value of the received signal is compared to a threshold (TH). The threshold can be indicative of a threshold distance of the telemetry coil from an external telemetry coil. A value of the input signal above the threshold can indicate that a distance of the telemetry coil from the external telemetry coil does not exceed the threshold distance, whereas the value of the input signal at or below the threshold can indicate that the distance of the telemetry coil from the external telemetry coil meets or exceeds the threshold distance.

If the input signal is above the threshold, a single-ended mode of operation can be determined at step 403 and a transmitter circuit can drive the telemetry coil in the single-ended mode. If the input signal is at or below the threshold, a differential mode can be determined at step 404 and the transmitter circuit can drive the telemetry coil in the differential mode.

Although illustrated as a series of steps above, in certain examples, one or more steps are optional, and in other examples, different combinations or permutations of these or other steps or examples can be combined to form other methods or processes, which is also applicable to other examples discussed herein.

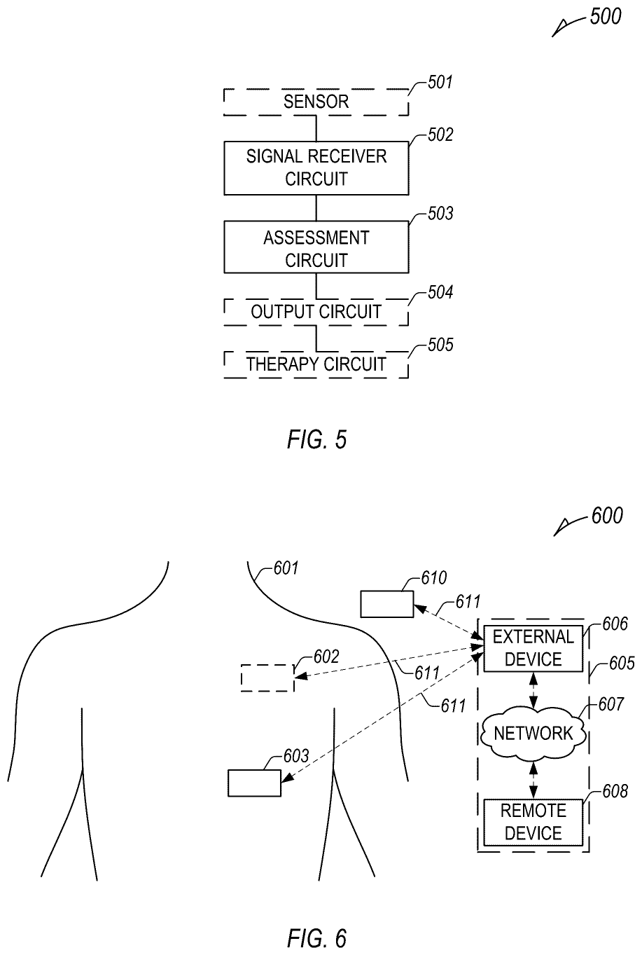

FIG. 5 illustrates an example system 500 (e.g., a medical device system). In an example, one or more aspects of the system 500 can be a component of, or communicatively coupled to, a medical device, such as an implantable medical device (IMD), an insertable cardiac monitor, an ambulatory medical device (AMD), etc. The system 500 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 500 can include a single medical device or a plurality of medical devices implanted in a body of a patient or otherwise positioned on or about the patient to monitor patient physiologic information of the patient using information from one or more sensors, such as a sensor 501. In an example, the sensor 501 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 500 can include a signal receiver circuit 502 and an assessment circuit 503. The signal receiver circuit 502 can be configured to receive physiologic information of a patient (or group of patients) from the sensor 501. The assessment circuit 503 can be configured to receive information from the signal receiver circuit 502, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 502 can include the sensor 501. In other examples, the signal receiver circuit can be coupled to or a component of the assessment circuit 503.

In certain examples, the assessment circuit 503 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 500 can include an output circuit 504 configured to provide an output to a user, or to cause an output to be provided to a user, such as through an output, a display, or one or more other user interface, the output including a score, a trend, an alert, or other indication. In other examples, the output circuit 504 can be configured to provide an output to another circuit, machine, or process, such as a therapy circuit 505 (e.g., a cardiac resynchronization therapy circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more cardiac resynchronization therapy parameters, drug delivery, dosage determinations or recommendations, etc. In an example, the therapy circuit 505 can include one or more of a stimulation control circuit, a cardiac stimulation circuit, a neural stimulation circuit, a dosage determination or control circuit, etc. In other examples, the therapy circuit 505 can be controlled by the assessment circuit 503, or one or more other circuits, etc. In certain examples, the assessment circuit 503 can include the output circuit 504 or can be configured to determine the output to be provided by the output circuit 504, while the output circuit 504 can provide the signals that cause the user interface to provide the output to the user based on the output determined by the assessment circuit 503.

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate (RR), tidal volume (TV), rapid shallow breathing index (RSBI), respiration phase, breathing sounds, etc.), etc. In one example, heart sounds and patient activity can be detected using non-overlapping time periods of the same, single- or multi-axis accelerometer, at different sampling frequencies and power costs. In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

Indications of patient condition (e.g., improved or worsening patient condition, etc.) can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

FIG. 6 illustrates an example patient management system 600 and portions of an environment in which the patient management system 600 may operate. The patient management system 600 can perform a range of activities, including remote patient monitoring and diagnosis of a disease condition, programming of ambulatory medical devices, and control of one or more therapies. Such activities can be performed proximal to a patient 601, such as in a patient home or office, through a centralized server, such as in a hospital, clinic, or physician office, or through a remote workstation, such as a secure wireless mobile computing device.

The patient management system 600 can include one or more medical devices, an external system 605, and a communication link 611 providing for communication between the one or more ambulatory medical devices and the external system 605. The one or more medical devices can include an ambulatory medical device, such as an implantable medical device 602, a wearable medical device 603, or one or more other implantable, leadless, subcutaneous, external, wearable, or medical devices configured to monitor, sense, or detect information from, determine physiologic information about, or provide one or more therapies to treat various conditions of the patient 601, such as one or more cardiac or non-cardiac conditions (e.g., dehydration, sleep disordered breathing, etc.).

In an example, the implantable medical device 602 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 601. In another example, the implantable medical device 602 can include a monitor implanted, for example, subcutaneously in the chest of patient 601, the implantable medical device 602 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers, small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemakers can communicate between themselves, or one or more other implanted or external devices.

The implantable medical device 602 can include a signal receiver circuit or an assessment circuit configured to detect or determine specific physiologic information of the patient 601, or to determine one or more conditions or provide information or an alert to a user, such as the patient 601 (e.g., a patient), a clinician, or one or more other caregivers or processes, such as described herein. The implantable medical device 602 can alternatively or additionally be configured as a therapeutic device configured to treat one or more medical conditions of the patient 601. The therapy can be delivered to the patient 601 via the lead system and associated electrodes or using one or more other delivery mechanisms. The therapy can include delivery of one or more drugs to the patient 601, such as using the implantable medical device 602 or one or more of the other ambulatory medical devices, etc. In some examples, therapy can include cardiac resynchronization therapy for rectifying dyssynchrony and improving cardiac function in heart failure patients. In other examples, the implantable medical device 602 can include a drug delivery system, such as a drug infusion pump to deliver drugs to the patient for managing arrhythmias or complications from arrhythmias, hypertension, hypotension, or one or more other physiologic conditions. In other examples, the implantable medical device 602 can include one or more electrodes configured to stimulate the nervous system of the patient or to provide stimulation to the muscles of the patient airway, etc.

The wearable medical device 603 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 605 can include a dedicated hardware/software system, such as a programmer, a remote server-based patient management system, or alternatively a system defined predominantly by software running on a standard personal computer. The external system 605 can manage the patient 601 through the implantable medical device 602 or one or more other ambulatory medical devices connected to the external system 605 via a communication link 611. In other examples, the implantable medical device 602 can be connected to the wearable medical device 603, or the wearable medical device 603 can be connected to the external system 605, via the communication link 611. This can include, for example, programming or reprogramming the implantable medical device 602 with different parameter settings to perform one or more of acquiring physiologic data, performing at least one self-diagnostic test (such as for a device operational status), analyzing the physiologic data, or optionally delivering or adjusting a therapy for the patient 601. Additionally, the external system 605 can send information to, or receive information from, the implantable medical device 602 or the wearable medical device 603 via the communication link 611. Examples of the information can include real-time or stored physiologic data from the patient 601, diagnostic data, such as detection of patient hydration status, hospitalizations, responses to therapies delivered to the patient 601, or device operational status of the implantable medical device 602 or the wearable medical device 603 (e.g., battery status, lead impedance, etc.). The communication link 611 can be an inductive telemetry link, a capacitive telemetry link, or a radio frequency (RF) telemetry link, such as a wireless telemetry based on, for example, Bluetooth® or IEEE 802.11 wireless fidelity “Wi-Fi” interfacing standards. Other configurations and combinations of patient data source interfacing are possible.

The external system 605 can include an external device 606 in proximity of the one or more ambulatory medical devices, and a remote device 608 in a location relatively distant from the one or more ambulatory medical devices, in communication with the external device 606 via a communication network 607. Examples of the external device 606 can include a medical device programmer. The remote device 608 can be configured to evaluate collected patient or patient information and provide alert notifications, among other possible functions. In an example, the remote device 608 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 608 can receive data from multiple patients. The data can be collected by the one or more ambulatory medical devices, among other data acquisition sensors or devices associated with the patient 601. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 608 may additionally include one or more locally configured clients or remote clients securely connected over the communication network 607 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 608, including the server and the interconnected clients, may also execute a follow-up scheme by sending follow-up requests to the one or more ambulatory medical devices, or by sending a message or other communication to the patient 601 (e.g., the patient), clinician or authorized third party as a compliance notification.

The communication network 607 can provide wired or wireless interconnectivity. In an example, the communication network 607 can be based on the Transmission Control Protocol/Internet Protocol (TCP/IP) network communication specification, although other types or combinations of networking implementations are possible. Similarly, other network topologies and arrangements are possible.

One or more of the external device 606 or the remote device 608 can output the detected medical events to a system user, such as the patient or a clinician, or to a process including, for example, an instance of a computer program executable in a microprocessor. In an example, the process can include an automated generation of a programming recommendation for an ambulatory medical device to improve cardiac capture for the patient. In an example, the external device 606 or the remote device 608 can include a respective display unit for displaying the physiologic or functional signals, or alerts, alarms, emergency calls, or other forms of warnings to signal the detection of one or more conditions. In some examples, the external system 605 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 602, the wearable medical device 603, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 608, or via a user interface of a software application executing on a client device communicatively connected with the remote device 608. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 605 can be implemented using hardware, software, firmware, or combinations thereof. Portions of the one or more ambulatory medical devices or the external system 605 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 610 can be configured to send information to or receive information from one or more of the ambulatory medical devices or the external system 605 using the communication link 611. In an example, the one or more ambulatory medical devices, the external device 606, or the remote device 608 can be configured to control one or more parameters of the therapy device 610. The external system 605 can allow for programming or reprogramming the one or more ambulatory medical devices and can receive information about one or more signals acquired by the one or more ambulatory medical devices, such as can be received via a communication link 611. The external system 605 can include a local external implantable medical device programmer. The external system 605 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite first heart sound (S1) parameter representative of a plurality of S1 parameters, for example, over a certain time period (e.g., a number of cardiac cycles, a representative time period, etc.), or one or more other heart sounds (e.g., a second heart sound (S2), a third heart sound (S3), a fourth heart sound (S4), etc.), etc.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient. In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia event, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

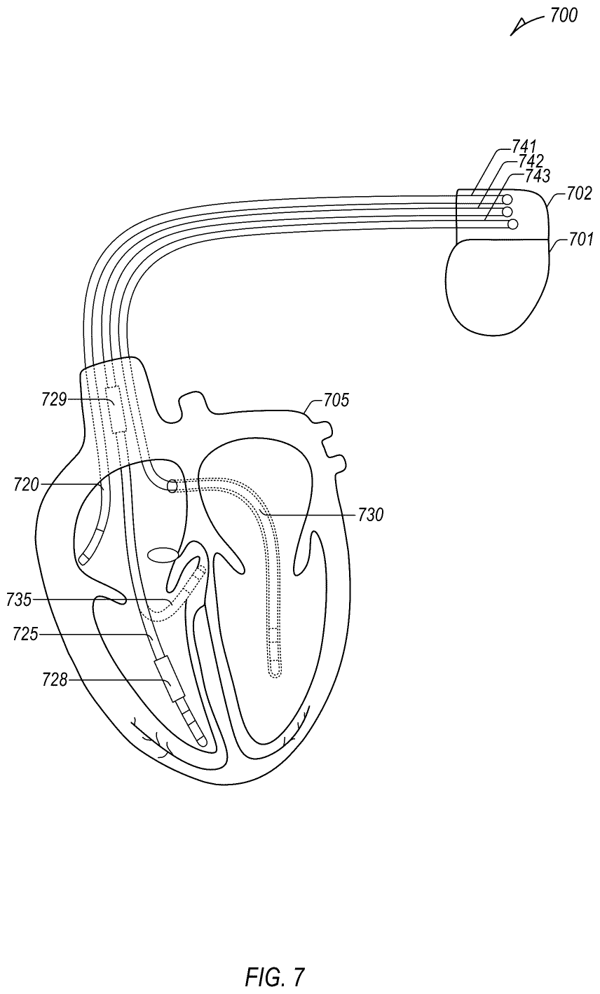

FIG. 7 illustrates an example implantable medical device system 700 including an implantable medical device 701 electrically coupled to a heart 705, such as through one or more leads coupled to the implantable medical device 701 through one or more lead ports, including first, second, or third lead ports 741, 742, 743 in a header 702 of the implantable medical device 701. In an example, the implantable medical device 701 can include an antenna, such as in the header 702, configured to enable communication with an external system and one or more electronic circuits (e.g., an assessment circuit, etc.) in a hermetically sealed housing (CAN).

The implantable medical device 701 may include an implantable cardiac monitor, pacemaker, defibrillator, cardiac resynchronization therapy device, or other subcutaneous implantable medical device or cardiac rhythm management (CRM) device configured to be implanted in a chest of a subject, having one or more leads to position one or more electrodes or other sensors at various locations in or near the heart 705, such as in one or more of the atria or ventricles. Separate from, or in addition to, the one or more electrodes or other sensors of the leads, the implantable medical device system 700 can include one or more electrodes or other sensors (e.g., a pressure sensor, an accelerometer, a gyroscope, a microphone, etc.) powered by a power source in the implantable medical device 701. The one or more electrodes or other sensors of the leads, the implantable medical device 701, or a combination thereof, can be configured detect physiologic information from, or provide one or more therapies or stimulation to, the patient.

Implantable devices can additionally include a leadless cardiac pacemaker, small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 705 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 701 can include one or more electronic circuits configured to sense one or more physiologic signals, such as an electrogram or a signal representing mechanical function of the heart 705. In certain examples, the housing may function as an electrode such as for sensing or pulse delivery. For example, an electrode from one or more of the leads may be used together with the housing such as for unipolar sensing of an electrogram or for delivering one or more pacing pulses. A defibrillation electrode (e.g., the first defibrillation coil electrode 728, the second defibrillation coil electrode 729, etc.) may be used together with the housing to deliver one or more cardioversion/defibrillation pulses.

In an example, the implantable medical device 701 can sense impedance such as between electrodes located on one or more of the leads or the housing. The implantable medical device 701 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 701 can be configured to inject current between an electrode on one or more of the first, second, third, or fourth leads 720, 725, 730, 735 and the housing, and to sense the resultant voltage between the same or different electrodes and the housing.

The implantable medical device 701 can integrate one or more other physiologic sensors to sense one or more other physiologic signals, such as one or more of heart rate, heart rate variability, thoracic or intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are.

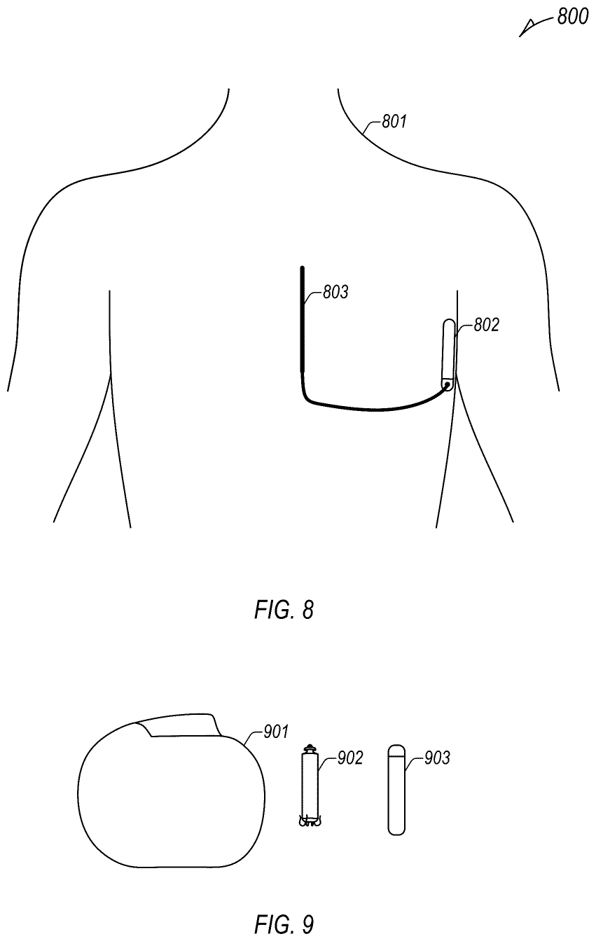

FIG. 8 illustrates an example implantable medical device system 800 including a subcutaneous implantable cardioverter defibrillator 802 implanted at a lateral thoracic region of a patient 801 and a subcutaneous lead 803 including a defibrillation coil and proximal and distal sensing electrodes implanted subcutaneously along a thorax of the patient 801.

FIG. 9 illustrates example implantable medical devices, including a subcutaneous implantable cardioverter defibrillator 901, a leadless cardiac pacemaker 902 including tines for placement, and an insertable cardiac monitor 903.

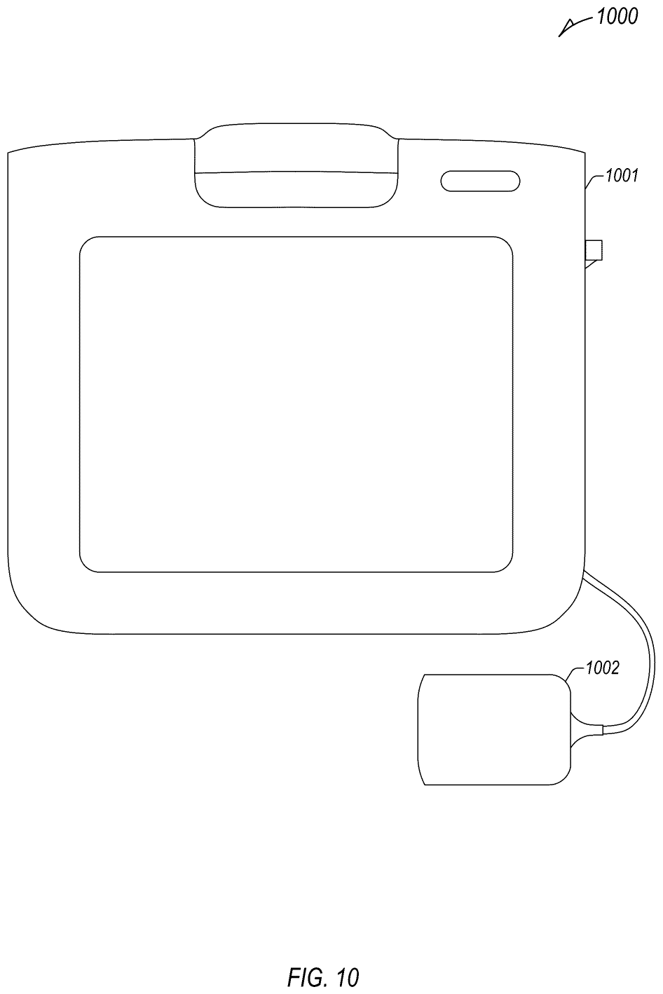

FIG. 10 illustrates an example remote patient management system 1000 including a remote patient management device 1001 (e.g., LATITUDE™ Programming System, Model 3300, etc.) and an external telemetry wand 1002 (e.g., a Model 6395 Telemetry Wand, etc.) including an external telemetry coil configured for inductive communication with a corresponding telemetry coil of an implantable medical device.

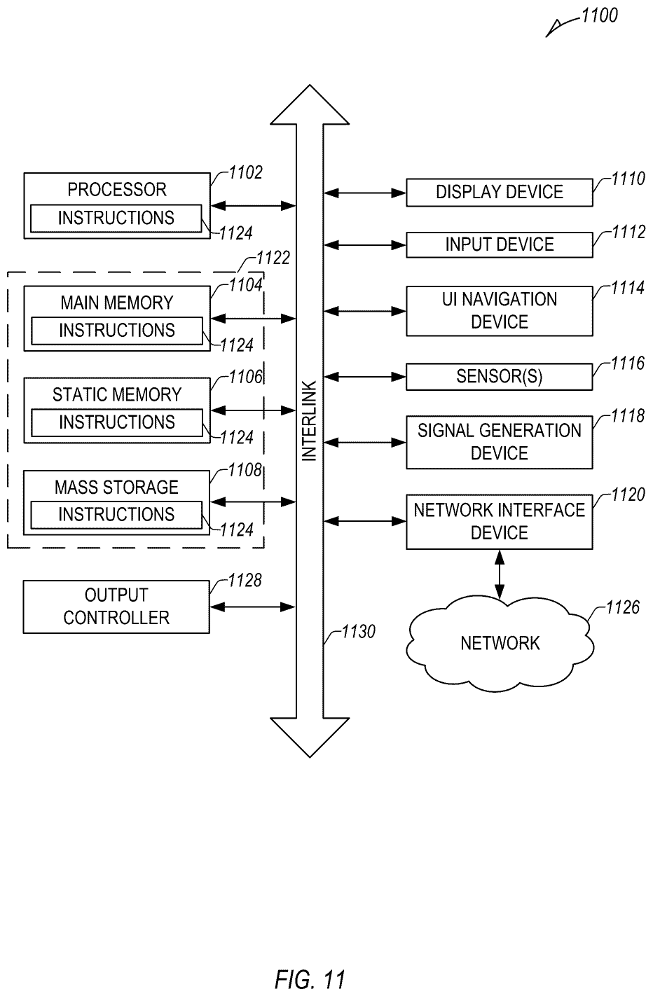

FIG. 11 illustrates a block diagram of an example machine 1100 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1100. Circuitry (e.g., processing circuitry, an assessment circuit, etc.) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to perform a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to perform portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may function as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1100 (e.g., computer system) may include a hardware processor 1102 (e.g., a central processing unit (CPU), a graphics processing unit (GPU), a hardware processor core, or any combination thereof), a main memory 1104, a static memory 1106 (e.g., memory or storage for firmware, microcode, a basic-input-output (BIOS), unified extensible firmware interface (UEFI), etc.), and mass storage 1108 (e.g., hard drive, tape drive, flash storage, or other block devices) some or all of which may communicate with each other via an interlink 1130 (e.g., bus). The machine 1100 may further include a display unit 1110, an alphanumeric input device 1112 (e.g., a keyboard), and a user interface (UI) navigation device 1114 (e.g., a mouse). In an example, the display unit 1110, input device 1112, and UI navigation device 1114 may be a touch screen display. The machine 1100 may additionally include a signal generation device 1118 (e.g., a speaker), a network interface device 1120, and one or more sensors 1116, such as a global positioning system (GPS) sensor, compass, accelerometer, or one or more other sensors. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine-readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine-readable medium 1122. While the machine-readable medium 1122 is illustrated as a single medium, the term “machine-readable medium” may include a single medium or multiple media (e.g., a centralized or distributed database, and/or associated caches and servers) configured to store the one or more instructions 1124.

The term “machine-readable medium” may include any medium that is capable of storing, encoding, or carrying instructions for execution by the machine 1100 and that cause the machine 1100 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1100, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. An implantable medical device system configured for inductive telemetry, comprising:
a receiver circuit configured to receive an input signal from a telemetry coil; and
a transmitter circuit having a single-ended mode and a differential mode, wherein the transmitter circuit is configured to selectively drive the telemetry coil in one of the single-ended mode or the differential mode using information from the receiver circuit.

2. The implantable medical device system of claim 1, comprising:
a control circuit configured to determine a mode of operation for the transmitter circuit using information from the receiver circuit,
wherein the transmitter circuit is configured to drive the telemetry coil in one of the single-ended mode or the differential mode based on the determined mode of operation.

3. The implantable medical device system of claim 1, wherein the transmitter circuit is configured to drive the telemetry coil in the single-ended mode if the information from the receiver circuit indicates that a distance of the telemetry coil from an external telemetry coil does not exceed a threshold distance.

4. The implantable medical device system of claim 1, wherein the transmitter circuit is configured to drive the telemetry coil in the differential mode if the information from the receiver circuit indicates that a distance of the telemetry coil from an external telemetry coil exceeds a threshold distance.

5. The implantable medical device system of claim 1, wherein the receiver circuit includes a comparator having a dynamic range and one of an amplifier or a resistor network, and
wherein the receiver circuit is configured to determine an attenuation or gain setting for the receiver circuit to apply to the input signal from the telemetry coil using the amplifier or the resistor network to keep an attenuated or amplified input signal in the dynamic range of the comparator.

6. The implantable medical device system of claim 5, wherein the dynamic range of the comparator has high and low thresholds,
wherein the receiver circuit is configured to adjust the attenuation or gain setting for the receiver circuit using the attenuated or amplified input signal exceeding the high threshold or falling below the low threshold,
wherein the attenuation or grain setting comprises at least two settings, a first setting indicating more attenuation and a second indicating less attenuation, and
wherein the transmitter circuit is configured to drive the telemetry coil in the single-ended mode in the first setting and in the differential mode in the second setting.

7. The implantable medical device system of claim 1, wherein the transmitter circuit is configured to transition from the single-ended mode to the differential mode, or from the differential mode to the single-ended mode, using information from the receiver circuit.

8. The implantable medical device system of claim 1, wherein to drive the telemetry coil in the single-ended mode comprises to drive the telemetry coil between a supply voltage of the implantable medical device and ground.

9. The implantable medical device system of claim 1, wherein to drive the telemetry coil in the differential mode comprises to drive the telemetry coil between a supply voltage of the implantable medical device and a negative supply voltage of the implantable medical device.

10. The implantable medical device system of claim 1, wherein the information from the receiver circuit includes a current or a voltage produced by the telemetry coil in response to an applied electromagnetic field by an external telemetry coil.

11. The implantable medical device system of claim 1, comprising a telemetry block including the receiver circuit, the transmitter circuit, and the telemetry coil, wherein the telemetry coil comprises a coil of wire having a number of turns,
wherein the telemetry block is configured to communicate digital data between the implantable medical device and a remote patient management system comprising an external telemetry coil.

12. A method for inductive telemetry in an implantable medical device, comprising:
receiving an input signal from a telemetry coil using a receiver circuit; and
selectively driving the telemetry coil, using a transmitter circuit having a single-ended mode and a differential mode, in a selective one of the single-ended mode or the differential mode using information from the receiver circuit.

13. The method of claim 12, comprising:
determining a mode of operation for the transmitter circuit using information from the receiver circuit,
wherein selectively driving the telemetry coil includes driving the telemetry coil in one of the single-ended mode or the differential mode based on the determined mode of operation.

14. The method of claim 12, wherein selectively driving the telemetry coil includes driving the telemetry coil in the single-ended mode if the information from the receiver circuit indicates that a distance of the telemetry coil from an external telemetry coil does not exceed a threshold distance.

15. The method of claim 12, wherein selectively driving the telemetry coil includes driving the telemetry coil in the differential mode if the information from the receiver circuit indicates that a distance of the telemetry coil from an external telemetry coil exceeds a threshold distance.

16. The method of claim 12, comprising:
determining an attenuation or gain setting for the receiver circuit to apply to the input signal from the telemetry coil using one of an amplifier or a resistor network to keep an attenuated or amplified input signal in a dynamic range of a comparator of the receiver circuit.

17. The method of claim 16, comprising:
adjusting the attenuation or gain setting for the receiver circuit using the attenuated or amplified input signal exceeding a high threshold or falling below a low threshold,
wherein selectively driving the telemetry coil includes driving the telemetry coil in the single-ended mode in a first attenuation or grain setting indicating more attenuation and in the differential mode in a second attenuation or grain setting indicating less attenuation.

18. The method of claim 12, comprising:
transitioning the transmitter circuit from the single-ended mode to the differential mode, or from the differential mode to the single-ended mode, using information from the receiver circuit.

19. The method of claim 12, wherein driving the telemetry coil in the single-ended mode comprises driving the telemetry coil between a supply voltage of the implantable medical device and ground, and
wherein driving the telemetry coil in the differential mode comprises driving the telemetry coil between the supply voltage of the implantable medical device and a negative supply voltage of the implantable medical device.

20. The method of claim 12, comprising:
communicating digital data in an inductive telemetry session between the implantable medical device and a remote patient management system,
wherein the implantable medical device comprises a telemetry block including the receiver circuit, the transmitter circuit, and the telemetry coil, wherein the telemetry coil comprises a coil of wire having a number of turns,
wherein the remote patient management system comprises an external telemetry coil, and
wherein the information from the receiver circuit includes a current or a voltage produced by the telemetry coil in response to an applied electromagnetic field by the external telemetry coil.
